(19) United States
(12) Patent Application Publication
(10) Pub. No.: US 2011/0230772 A1
(43) Pub. Date: Sep. 22, 2011

(54) METHOD AND DEVICE FOR INVASIVE BLOOD PRESSURE MEASUREMENT IN A VASCULAR ACCESS

(75) Inventors: Sebastian Koball, Rostock (DE); Heiko Hickstein, Kritzmow (DE); Jan Stange, Rostock (DE); Steffen Mitzner, Rostock (DE)

(73) Assignee: Cellumetrix UG, Rostock (DE)

(21) Appl. No.: 13/131,721

(22) PCT Filed: Nov. 26, 2009
(86) PCT No.: PCT/EP2009/065880

§ 371 Date: May 27, 2011

(30) Foreign Application Priority Data
Nov. 27, 2008 (DE) 10 2008 059 379.6

(51) Int. Cl. A61B 5/021; A61M 1/14

(52) U.S. Cl. 600/485; 604/4.01

(57) ABSTRACT

A device and method for invasive blood pressure measurement in a vascular access under continuous blood flows in a treatment device in extracorporeal detoxification methods is provided. Systemic arterial pressure is directly measured using an existing vascular access for dialysis, or in which the systemic arterial pressure and the temporal progression of such pressure are determined indirectly. A valve-controlled bypass system which goes around a blood pumping unit is provided so that the blood flow in the treatment device is not interrupted, and alarms are suppressed The bypass module is easily connectable to measuring equipment on various treatment units without having to adjust the same.

### BACKGROUND OF THE INVENTION

The invention relates to a device and to a method for invasive blood pressure measurement in a vascular access under continuous or discontinuous blood flow in a treatment device in extracorporeal detoxification methods.

Dialysis therapy is an established, well known treatment option for patients with an acute or chronic renal failure. Different options in broader sense (hemodialysis, hemofiltration, hemodiafiltration) replace the function of the kidney, e.g. detoxification and excretion.

The Kidneys play a cautious role in the regulation of body water and solute concentrations and the excretion of water soluble metabolism products and other substances. These therapy options imitate the natural detoxification and excretion function of the kidneys.

One great disadvantage of the actual renal replacement therapy is the limitation of treatment time in intermittent treatment schemes. Patients on chronic renal replacement therapy are treated usually three times a week for 4 to 5 hours (12-15 hours/week). In comparism to the normal kidney function (24 hours/day and 7 days/week=168 hours/week) there is a significantly reduced time for removing toxic substances and water from a patient's body. Patients with an end stage renal failure without any urine production often suffer from a chronic fluid overload.

Due to this fluid overload, the cardiovascular system is stressed. The heart must pump more blood volume against a higher systemic blood pressure in the patient's body. Edemas in peripheral body parts are the result. Fluid accumulation in the lungs leads to severe shortness of breath. One task of the kidney in healthy people or the dialysis treatment in patients is to regulate the fluid balance.

Healthy people have a daily fluid intake of approximately 2 to 2.5 liters a day. This leads to an urine production of 1.2 to 1.5 liters a day. Further fluid loss is due to breathing and sweat. The body weight as a target value of regulations stays constant. The kidneys are able to produce more or less concentrated urine depending on whole body fluid load.

Patients with a chronic renal failure have an daily fluid intake of approximately 1 liter. The fluid volume for drinking is limited to 600-800 ml/day. Another problem is an impaired feeling of thirst leading to excessive fluid intake despite fluid overload. But the patients body is not able to increase urine volume. Therefore, in every treatment session a fluid removal is necessary. To get the body weight and the fluid balance at an optimal level, in every treatment session 5-6 liters of water have to be removed by the dialysis machine. This fluid removal is burdensome for the patient and has a high risk for circulation failure and a depression of blood pressure. This can occur during or after a treatment session, leading to a dysfunction of other organs.

Disturbances of blood pressure and circulation during renal replacement therapy are uncomfortable and risky for the patient. Nausea, dizziness, vomiting, cramps and other severe complications like unconsciousness and heart arrest are common complications during dialysis sessions. It is proven that this complications decrease the life expectancy of the patients.

For prevention of circulation failure and decreasing blood pressure during dialysis treatment, the patient should avoid high drinking volumes.

Now it is usual for the prevention of severe problems and the early detection of such complications to measure the blood pressure according to the method of Riva-Cocci with a pneumatic inflatable cuff and a manometer. The frequency of blood pressure measurement is adjusted to the personal risk profile of each patient. A drop down of blood pressure often occurs suddenly without any premonition. This is difficult for early and timely reaction and treatment of complications.

The treatment of blood pressure depression is possible using infusion of fluids, stopping fluid removal, prolonging dialysis time or infusion of salt solutions. Critical is the early recognition of decreasing blood pressure. Using the conventional blood pressure monitoring the measurement gap is often too long for an early detection of circulation failure. Shortening the interval between two conventional blood pressure measurements is often not tolerated by the patient, because this may be uncomfortable or painful.

Attempts to avoid these problems are known, but all of them show some inadequacies or difficulties. A broad application of these techniques does not take place to date.

It is possible to shorten the measurement interval for the early detection of decreasing blood pressure values. The use of algorithms for the prediction of the course of blood pressure is also known (Fuzzy logic controlled, DE 19821534 C1). The use of non invasive conventional blood pressure measurement method with a long measurement interval is not sufficient for the prediction of course of blood pressure. The use of data bases with documented blood pressure courses of former treatment sessions is also not sufficient, especially in case of emergency dialysis sessions.

The non-invasive measurement of other circulation parameters is known. (DE 19746377 C1, DE 102006010813 A1, WO 2006031186 A1) These techniques are often not sufficient because of the high heterogeneity of the patient clientel and the stiffness of their vessels.

In intensive care medicine, continuous invasive blood pressure measurement is applied. This requires a special arterial vascular access, such an intra arterial catheter. The installation of an arterial catheter is costly and time intensive and risky for the patient, so that this no alternative for the regular monitoring of chronic dialysis patients.

The measurement of the pressure in the vascular access and in the blood lines of the therapy unit is performed for monitoring the connectivity from the patient and the dialysis machine and for monitoring the blood flow and the functional state of the vascular access (DE 19901078 C1, DE 19734002 C1, EP 1584339 A2). Such a mannered unit is essential in every dialysis machine for detecting a loss of connectivity between patient and therapy unit.

The pressure measurement is performed during the running of blood pumps, so that a determination of the systemic blood pressure of the patient is not possible. An extension of these procedures with measurement of the fistula pressure during different speeds of the blood pump for the adaptation and improvement of the optimal blood flow rates are also known (DE 10254988 B4, DE 10112848 A1). But these methods are also not able to determine a patient's systemic blood pressure (Jacobs/Kjellestrand/Koch/Winchester, Replacement of renal function by dialysis, 4th Edition).

Devices for the measurement of blood pressure in the vascular access are also known in different forms (DE 69203249 T2, CA 2235601 A1). These measurements take place for monitoring the therapy, as well as for the monitoring of a patient's blood pressure. If the measurements take place for the monitoring of patients blood pressure or for the absolute determination of the fistula pressure, however, the measurements are always connected with a hold of the blood pump (DE 10254988 B4). However, this shows some disadvantages. By the break of the blood flow in the device it comes to activation of coagulation and with it to one blocking of the dialysis filter and blood lines. The therapy monitoring equipment of the therapy device recognize the hold of the blood flow and stop the whole treatment. For the adaptation of the measuring principle (invasive pressure measurement in the dialysis access) to different manufacturers of therapy devices, fundamental interventions would be necessary in the control system of each dialysis machine.

A device for the invasive measurement of the blood flow in the fistula is known (U.S. Pat. No. 6,746,408 B2). But with this method, there is no conclusion to the systemic blood pressure of the patient.

### BRIEF SUMMARY OF THE INVENTION

The present invention provides a procedure and a device for monitoring a patient's blood pressure. Therefore the vascular access (fistula) will be used for measuring the pressure in the fistula to make decisions to the absolute value and the time course of the systemic blood pressure of the patient.

The procedure avoids all disadvantages of the state of the actual technology by use of a valve-controlled bypass system which avoids a blood pump's hold. With it, the blood flow is not interrupted in the therapy device, and false alarms are suppressed. A connection of the bypass module and the measuring equipment is possible with different therapy units and brands. The device can be implemented into existing dialysis machines or as a stand alone device which can be adapted to the dialysis machine.

The procedure according to the invention is distinguished by the fact that a change of the blood flow is executed by a bypass procedure with a clamp system in the arterial and venous accesses of the patient and in a short circuit connection between both accesses. A measurement of the absolute values of the blood pressure, the steepness of the increase of pressure, the middle arterial blood pressure values, the reaching of a plateau phase and the increase of the definitely measured blood pressure values is possible, as well as the changes of the pulse volume curves in the vessel access during discontinuing the blood flow in the vascular access. The procedure is further distinguished by the fact that the blood flow in the therapy device is not interrupted and the fact that an electronic measurement and evaluation system monitors the changes of the measured values after change-over in the bypass procedure up to a plateau phase.

In the procedure of the present invention, the blood pressure measurement is performed at the existing (for chronic dialysis therapy) vascular access with a pressure converter connected to the blood lines of the therapy unit. The measuring signal is processed electronically and is sent to an evaluation unit. Differing from the invasive blood pressure measurement used in intensive care medicine, the measurement is not performed continuously, because a measurement is only possible during interrupted blood flow in the vascular access. This can be achieved by a short stop of the blood pump or by redirecting the blood flow into a bypass system. The bypass system allows a continued blood flow inside the dialysis machine without a stop of the blood pump, thus avoiding clotting problems in the blood lines or the dialysis filter. The distinction of the new invention is also marked by the bypass procedure using a new clamp control. By this clamp control, no intervention is necessary in the therapy unit itself. One advantage of this system is the easy adaptation of the measuring procedure to dialysis machines of different manufacturers. A combination or integration of the control unit of the bypass system into the control unit of the therapy unit is possible.

The measuring time can be kept short, so that the whole dialysis time is extended only insignificantly. A central component of the invention is the new combination of the known measuring procedures with the new principle of the blood flow redirection in a bypass system, as well as the procedure of the electronic evaluation of the measured values. The measurement of different dynamic parameters (slope of the arterial central pressure and the volume pulse in the slope phase) differs from the measurements obtained previously. The application of a bypass system which is steered by the application of clamps brings crucial advantages. The blood flow in the therapy unit is not interrupted and the risk of coagulation is avoided. Another advantage of the procedure of the present invention is that by using the bypass system no interventions are necessary in the dialysis machine itself (particularly in the monitoring and the control of the blood pump function). With the new system, it is also possible to perform a problem-free implementation into devices of different manufacturers, without colliding with the respective device directives.

Furthermore, as a new measurement is taken, it is measured as the time passes after the halt of the blood pump or activation of the bypass system until the measured pressure values have reached a stable plateau. It arises that this time correlates with the functional status of the vascular access, the circulating blood volume and in particular with the heartbeat volume. In addition, the absolute values of the blood pressure, as well as the curves courses of the values during stopped blood flow in the vascular access are considered the analysis process.

A further advantage of the new procedure is that frequent measuring is possible during the therapy and that also by frequent measurement, the measured values are not influenced by the measurement procedure itself, like in the common method of Riva-Rocci. A good correlation of the blood pressure measured in the vascular access and the systemic arterial pressure has been shown. Even when the absolute values often did not exactly show the same values, a conclusion on the course of patient systemic blood pressure is possible. Other optimisation can occur therefore through course observations in the same patient over several therapy meetings through adaptation of the patient specific settings. By derivation of the above mentioned parameters, it is possible to raise the security of the patient in the dialysis by an improved supervision and to raise the efficacy of treatment. This is realized by the parameters directed control of filtration rates, temperature of the dialysis solution, sodium concentration of the dialysate or with the application of solutions with high osmolarity (glucose or sodium solutions).

The device of the present invention is thereby distinguished by the fact that in the blood lines to the vascular access a pressure receiver is arranged which is connected with an measuring and evaluation unit, so as to obtain the absolute values of the blood pressure, the slope of pressure up to the achievement of a plateau phase and the slope of the absolutely measured blood pressure values of every single heart beat in the vascular access or in the blood lines during interruption of the blood flow. The invention is also distinguished by the fact that a bypass steered with the valves 1, 2 and 3 does not interrupt the blood flow in the therapy device during the measurement. The invention is further distinguished by the fact that the activation of the bypass mode is realized by a clamp or valve system in the arterial and venous accesses positioned patient-sided and in a short circuit connection between these accesses and the fact that the measured values after activation of the bypass mode up to a plateau phase are considered by the evaluation unit.

The bypass is made possible with a H-formed disposable tube set.

The pressure sensors carry out the measurement of the pressure and the dynamic increase of the pressure in the vascular access in an interval after clamp activation of the arterial blood line. The measured values are used as a parameter for the peripheral volume flow (depending on heart beat volume) and the blood pressure.

The arterial and venous blood inlets are clamped and a short circuit connection (bypass) is opened on the machine side of the blood line system. This allows a pump driven circulation of the blood outside the patient in the blood lines of the therapy unit.

The evaluation unit processes the measured absolute systolic and diastolic values and the course of the arterial pressure curve. It considers the increase of pressure speed and pressure drop as well as the increasing pressure value after activation of the bypass system or a halt of the blood pump.

The clamp devices or a modified bypass system cancel for the time of the measurement the monitoring system of the dialysis machine, which controls the therapy supervision and monitoring of the arterial, venous or transmembrane pressure.

During the time of activated bypass system, the clamp system overrides the machine-sided pressure control to avoid false alarms.

The evaluation unit gives a signal or alarm, which allows an optimization or an adaptation of the actual treatment modalities to the actual treatment session. The evaluation unit stores the measured values to optimize the next treatment sessions, comparing stored values with actual values.

The evaluation unit monitors the measured and calculated values for better patient security during the extracorporeal treatment. The evaluation unit could also control the treatment course by an adaption of time line, ultrafiltration rates, dialysate temperature or the administration of fluid volumes or saline and glucose solutions.

The software of the control and evaluation units regulates the activation of clamps or valves for the activation of the bypass system in arterial, venous, and machine sided blood lines.

In the monitoring and supervision of the state of blood pressure and circulation in patients on extracorporeal treatment modalities, the system of the present invention. The system is contemplated to improve the security and tolerance of extracorporeal renal replacement therapy and avoid severe complications. The combination of the new described measuring procedure with existing regularization principles to the direct control of dialysis machines is also comtemplated.

### BRIEF DESCRIPTION OF THE DRAWINGS

FIG. 1 is a schematic arrangement of the device according to the present invention; and

FIG. 2 is a graphic representation of the parameters to be evaluated.

### DETAILED DESCRIPTION OF THE INVENTION

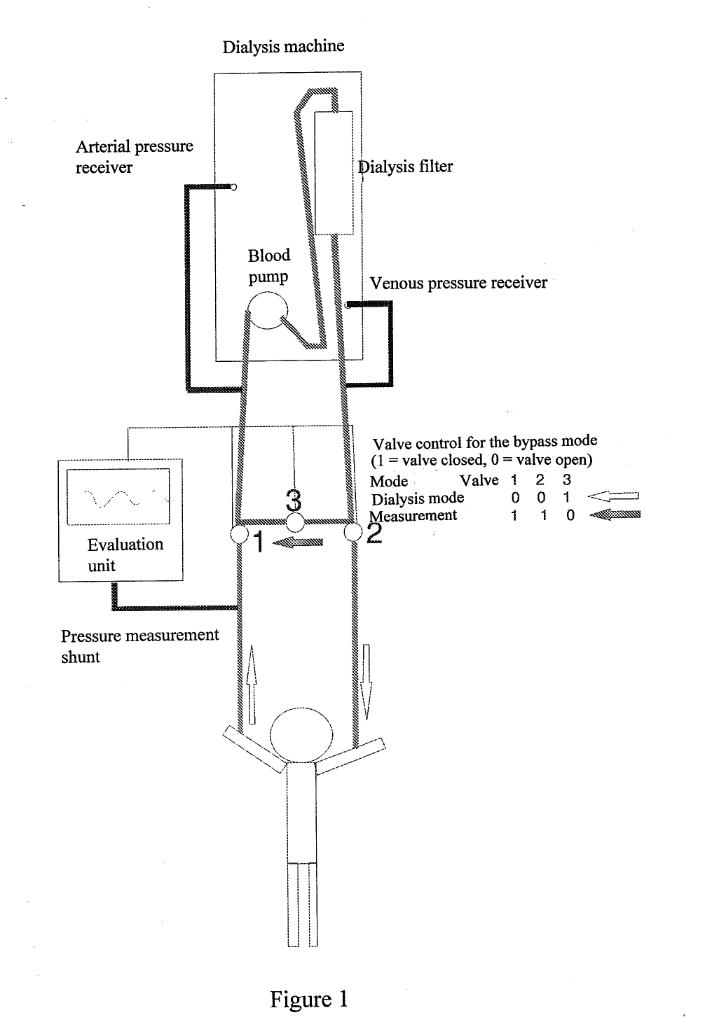

The schematic arrangement of the device of the invention in FIG. 1 shows a dialysis machine with the dialysis filter and the blood pump as well as an arterial and a venous pressure receiver. The pressure measurement according to the invention occurs in the vascular access during interruption of the blood flow in the vascular access, while the blood flow in the therapy device is redirected in the bypass system. The blood flow in the treatment device is not interrupted. Pressure measurement occurs in the vascular access while the bypass is activated. The bypass is steered about valves 1, 2 and 3. In the normal dialysis, mode the valve 1 is opened to the arterial pressure receiver and the valve 2 to the venous pressure receiver and the valve 3 which lies between venous and arterial pressure receiver is closed. During the measurement, the valves 1 and 2 are closed, while valve 3 is opened. The blood flow in the arterial and venous vascular accesses is interrupted. The blood flow in the treatment device is not interrupted, and is driven by the blood pump, because the bypass supplies the blood stream from the venous to the arterial part of the blood lines. The bypass is established by a H-shaped tube set (H-Disposable tube set).

The pressure in the vascular access and the dynamic increase of the pressure in the vascular access during the time after activation of the bypass mode are measured over a pressure receiver in the blood line system. From these direct measured values, other parameters are derived by calculation.

The measurement of the dynamic values (slope and time of increasing pressure) takes place during the activation of the bypass mode and during the increase of pressure in the vascular access. After the activation of the bypass mode, a certain time is necessary until the blood pressure values have reached a stable plateau. This time in the activated bypass mode is measured. Furthermore, the maximum pressure difference is measured between pressure in the vascular access at the beginning of the bypass mode and after pressure balance. The average increase of pressure speed/slope of every pulse is also determined, which is an indirect measure of the peripheral volume pulse.

Interruptions of the flow of the dialysis blood pump by pressure fluctuations and alarms in the arterial, venous or transmembrane pressure of the machine are prevented, as these pressure measurement units are functionally inactivated during the activation of the bypass mode for a short time. This can be established by additional tube clamps or valves in the tube lines of the pressure receivers. These clamps or valves can be controlled through the measuring unit (independent from the dialysis machine) or by a modification of the control unit of the dialysis machine.

An automated evaluation considers the measured absolute values of the blood pressure, the slope of the increase of pressure and in particular the increase of the absolutely measured blood pressure values after activation of the bypass mode up to a plateau phase. Not only the absolute systolic and diastolic values are measured and evaluated, but also the course of the arterial pressure curve concerning an increase of pressure speed and pressure drop calculated and evaluated by an evaluation unit. This analysis considers in addition, the increase of pressure after redirecting the blood flow in the bypass system. The increase of the pressure in the vascular access blood lines after activation of the bypass mode is used as a parameter for the peripheral volume flow thereby distinguishing over conventional methods whereby, this dynamic measured value is used for circulation monitoring. The measuring values are stored to be able to carry out optimization of further treatment sessions.

With excess of lower or upper limits of measured or calculated values, a signal is released or an intervention initiated for the adaptation of the modalities of the treatment directly to the course of treatment.

The measured values and the parameters that are computer analyzed are used to supervise patients safety and to steer the extracorporeal therapy. The steering of the therapy is based on adaptation of ultrafiltration rates, dialysate temperature and by automated application of volume or concentrates of glucose or electrolytes according to the measured values and parameters predicting the patient's needs.

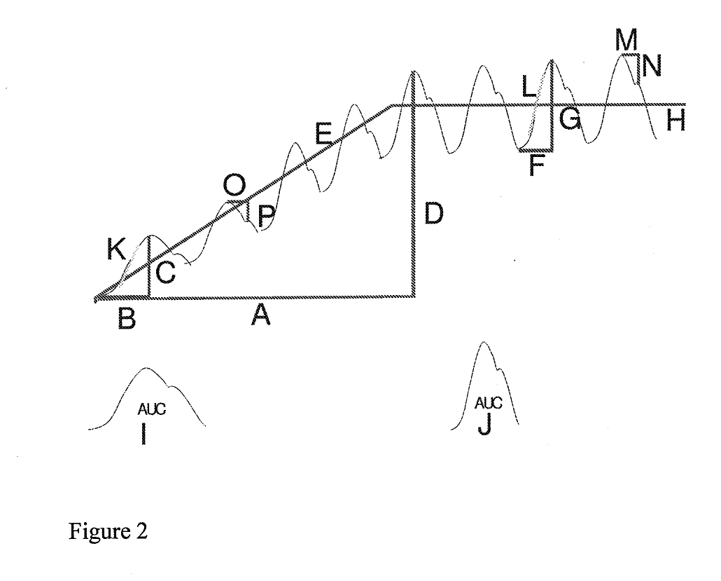

FIG. 2 depicts the values measured and the parameters thereof when computer analyzed. The measured pressure values (thin curve) are detected after activation of the bypass which leads to an increase of the pressures to a plateau. The parameters are:

A—time to plateau after activation of the bypass

D—maximum of the pressure difference between activation of the bypass and during the plateau.

E—pressure increase by time during activation of the bypass until plateau, an important indirect representation of the peripheral volume puls.

I and J are the areas under the curve for the pressure pulse at activation (I) and at plateau (J).

K and L represent the pressure increase by time from the minimum to the maximum pressure of a pulse curve at activation of the of the bypass (K) and at plateau (L).

B and F represent the time associated with K and L respectively. C and G represent the pressure increase associated with K and L respectively.

O and P and M and N describe the dicrotism of the pressure curve during activation of the bypass and at plateau.

1. A method for invasive blood pressure measurement in a vascular access, comprising:
activating a bypass mode so as to provide a bypass of arterial and venous dialysis tubes of a dialysis machine to allow the blood stream to continue while the pressures in the vascular access are measured under static conditions;
measuring the absolute values of the blood pressure between the bypass and the vascular access by invasive sensors from a time of activation of the bypass until a time that a plateau of the mean pressure is reached;
measuring the amplitudes of the blood pressure pulse curves from a time of activation of the bypass until a time that a plateau of the mean pressure is reached;
measuring the pulse volume curves from a time of activation of the bypass until a time that a plateau of the mean pressure is reached; and
computerizing analysis of changes of the above measured values and analyzing their change over time during activation of the bypass until a time when the plateau of the mean pressure is reached.

2. A process according to claim 1, comprising: using the pressures in the vascular access and the dynamic increase of the blood pressure as measured by sensors in the vascular access, after activation of the bypass, as parameters for peripheral volume flow and blood pressure.

3. A process according to claim 1, further comprising: interrupting blood flow out of the vascular access by using a valve; and activating a bypass so as to provide a bypass of the blood flow from the arterial and venous line on the machine side of the valve to allow the blood flow in the dialysis circuit to continue flowing during the measurements.

4. A process according to claim 1, comprising: measuring and analyzing absolute systolic and diastolic values, the time course of the arterial pressure curve, the increase of pressure over time after activation of the bypass, whereby the increase of the mean pressure after activation of the bypass is a representation of the peripheral volume stream.

5. A process according to claim 1, comprising bypassing sensors of the dialysis machine for arterial, venous and transmembrane pressure detection when the bypass is activated by valves or by modifying the bypass mode for pressure adaptation.

6. A process according to claim 1, comprising: using a system of valves to bypass machine pressure sensors in order to prevent false alarms on the machine side during the activation of the bypass.

7. A process according to claim 1, comprising: defining limits for measured values or calculated parameters and activating a signal once those limits are exceeded so as to initiate an adaptation of treatment parameters.

8. A process according to claim 1, comprising: storing said parameters to be used for optimization of further treatments.

9. A process according to claim 1, comprising: using measured values and calculated parameters for safety surveillance of the patient and for controlling the extracorporeal therapy by adaptation of ultrafiltration rates over time, adaptation of dialysate temperature, automated or manual application of volume, or concentrates of glucose or electrolytes.

10. A Process according to claim 1, wherein automated activation of arterial, venous, bypass and sensor blinding valves is controlled by software integrated in the dialysis machine.

11. A process according to claim 1, wherein automated activation of arterial, venous, bypass and sensor blinding valves is controlled by an external device not integrated in the dialysis machine.

12.-22. (canceled)

23. A bypass system and device for invasive blood pressure measurement in a vascular access, comprising:
a medical device attached to an arterial and a venous pressure sensor, that includes
a first valve capable of opening or closing an arterial blood tube line distal from a bypass,
a second valve capable of opening or closing a venous blood tube line distal from the bypass,
a third valve capable of opening or closing a bypass tube connecting the venous and arterial blood tubes proximate the first and second valves,
a pressure sensor positioned in the vascular access lines proximate to the bypass and the first and second valves and that measures the pressure inside the vascular access through use of a processing unit which is linked to this sensor and which processes the values and parameters from the time the bypass is activated until the pressures reach a plateau; and
valves that are positioned for (a) closing the arterial blood tube line distal from the bypass, (b) closing the venous blood tube line distal from the bypass, (c) closing the bypass tube connecting the venous and arterial blood tube proximal of the valves (a) and (b).

24. The bypass system and device according to claim 23, comprising an H-shaped disposable tube material that is used to create the bypass.

25. The bypass system and device according to claim 23, comprising an activation of the first, second and third valves so that such activation is achieved by the closing of the arterial blood tube line distal from the bypass, the closing of the venous blood tube line distal from the bypass, and the opening of the bypass tube connecting the venous and arterial blood tubes.

26. The bypass system and device according to claim 23, whereby the valves achieve overriding the pressure sensors of an extracorporeal device that is used to survey the arterial, venous or transmembrane pressure.

27. The bypass system and device according to claim 23, whereby the valves achieve overriding of the pressure sensors of an extracorporeal device that is used to survey the arterial, venous or transmembrane pressure in order to prevent false alarms.

28. The bypass system and device according to any one of claims 23-27, further comprising software integrated in the dialysis machine that controls the activation of the bypass by controlling the opening and closing of the valves.
